(19) United States
(12) Patent Application Publication
(10) Pub. No.: US 2025/0367130 A1
(43) Pub. Date: Dec. 4, 2025

(54) THERMORESPONSIVE HYDROGEL CONTAINING METAL-ORGANIC FRAMEWORK PARTICLES FOR NONINVASIVE OCULAR DRUG DELIVERY

(71) Applicant: University of Pittsburgh - Of the Commonwealth System of Higher Education, Pittsburgh, PA (US)

(72) Inventors: Phillip Alexander Harding, Murrysville, PA (US); Morgan Virginia DiLeo, Pittsburgh, PA (US)

(73) Assignee: University of Pittsburgh - Of the Commonwealth System of Higher Education, Pittsburgh, PA (US)

(21) Appl. No.: 19/222,303

(22) Filed: May 29, 2025

Related U.S. Application Data
(60) Provisional application No. 63/653,637, filed on May 30, 2024.
(60) Provisional application No. 63/728,386, filed on Dec. 5, 2024.

(51) Int. Cl. A61K 9/51; A61K 9/06; A61K 39/385

(52) U.S. Cl. CPC A61K 9/5123; A61K 9/06; A61K 39/385

(57) ABSTRACT

A composition that includes agent-loaded metal-organic framework nanoparticles dispersed within a thermoresponsive hydrogel, wherein the agent is an agent for treating an ocular condition and the composition is configured for sustained topical ocular release.

### CROSS REFERENCE TO RELATED APPLICATION

This application claims the benefit of and priority to U.S. Provisional Patent Application No. 63/653,637, filed May 30, 2024, and U.S. Provisional Patent Application No. 63/728,386, filed Dec. 5, 2024, both of which are incorporated herein by reference in its entirety.

### BACKGROUND

Non-inherited ocular pathologies, such as age-related macular degeneration and glaucoma, are the leading causes of blindess worldwide. Many risk factors play a role in the development of these diseases, such as hypertension, smoking, and age. Typical intervention methods for these diseases require frequent, daily, high dose administration of eye drops, or bimonthly injections into the retinal space. As a result, patient compliance rates are as low as 25% and disease progression continues.

It is estimated that nearly 4 million adults will be diagnosed with open angle glaucoma by the year 2020, the majority of which will be treated with a daily regimen of ocular hypotensive medication (Friedman et al., 2004). These IOP-reducing drugs are given as eye drops, which must be administered frequently by the patient to reduce the risk of irreversible vision loss. The rigorous dosing schedule, initial lack of symptoms, and difficult drop administration lead to extremely low patient compliance rates (Hermann et al., 2010). Additionally, eye drop administration requires high concentrations of drug to overcome the many absorption barriers in the eye (Ghate and Edelhauser, 2008).

Retinal diseases, such as age-related macular degeneration, diabetic retinopathy, and macular edema, are leading causes of vision impairment and loss worldwide (Blindness, G. B. D., C. Vision Impairment and S. Vision Loss Expert Group of the Global Burden of Disease (2021). “Trends in prevalence of blindness and distance and near vision impairment over 30 years: an analysis for the Global Burden of Disease Study.” Lancet Glob Health 9 (2): e130-e143.). For these diseases, anti-angiogenic molecules are prescribed and require monthly or bimonthly visits to the doctor's office for an intravitreal injection. As a result, patient compliance is low and loss-to-follow-up rates are high, with 50% discontinuing treatment over 5 years (Boulanger-Scemama, E., G. Querques, F. About, N. Puche, M. Srour, V. Mane, N. Massamba, F. Canoui-Poitrine and E. H. Souied (2015). “Ranibizumab for exudative age-related macular degeneration: A five year study of adherence to follow-up in a real-life setting.” J Fr Ophtalmol 38 (7): 620-627.) Eye drop administration has not been fully explored for treatment of retinal diseases.

Topical ocular drug delivery systems suffer from limitations to reach the retina, but approaches have been explored to overcome these limitations by addressing ocular residence time and applying controlled release strategies.

### SUMMARY

Disclosed herein is a composition comprising agent-loaded metal-organic framework nanoparticles dispersed within a thermoresponsive hydrogel, wherein the agent is an agent for treating an ocular condition and the composition is configured for sustained topical ocular release.

Also disclosed herein is a method comprising combining an agent with metal-organic framework nanoparticles; and combining the resulting agent-loaded metal-organic framework nanoparticles with a thermoresponsive hydrogel to form a composition.

Further disclosed herein is a method for ocular delivery of an agent comprising administering the agent at the lower fornix of an eye in a subject, wherein the method comprises topically delivering to an eye a liquid hydrogel comprising agent-loaded metal-organic framework nanoparticles, and permitting the liquid hydrogel to form in situ a gelled, sustained release structure residing in the lower fornix of the eye.

The foregoing will become more apparent from the following detailed description, which proceeds with reference to the accompanying figures.

### BRIEF DESCRIPTION OF THE DRAWINGS

FIGS. 1A-1C are a representation of an embodiment for administering an embodiment of the nanoparticle/hydrogel delivery system disclosed herein.

FIG. 2 is a graph showing cumulative mass of ZIF-8 based on fluorescence.

FIG. 3 is a graph showing cumulative release of comparative PLGA microparticles with the modification of loading from 1 mg to 10 mg.

FIG. 4 is a graph showing cumulative release of ZIF-8 metal-organic frameworks loaded with 1 g ovalbumin.

### DETAILED DESCRIPTION

### Terminology

The following explanations of terms and methods are provided to better describe the present compounds, compositions and methods, and to guide those of ordinary skill in the art in the practice of the present disclosure. It is also to be understood that the terminology used in the disclosure is for the purpose of describing particular embodiments and examples only and is not intended to be limiting.

An “animal” refers to living multi-cellular vertebrate organisms, a category that includes, for example, mammals and birds. The term mammal includes both human and non-human mammals. Similarly, the term “subject” includes both human and non-human subjects, including birds and non-human mammals, such as non-human primates, companion animals (such as dogs and cats), livestock (such as pigs, sheep, cows), as well as non-domesticated animals, such as the big cats.

The term “co-administration” or “co-administering” refers to administration of an agent disclosed herein with at least one other therapeutic or diagnostic agent within the same general time period, and does not require administration at the same exact moment in time (although co-administration is inclusive of administering at the same exact moment in time). Thus, co-administration may be on the same day or on different days, or in the same week or in different weeks. In certain embodiments, a plurality of therapeutic and/or diagnostic agents may be co-administered by encapsulating the agents within the nanoparticles disclosed herein.

“Inhibiting” refers to inhibiting the full development of a disease or condition. “Inhibiting” also refers to any quantitative or qualitative reduction in biological activity or binding, relative to a control.

“Nanoparticle”, as used herein, unless otherwise specified, generally refers to a particle of a relatively small size the term is used in reference to particles of sizes that can be, for example, administered to the eye in the form of an eye drop that can be delivered from a squeeze nozzle container and that can diffuse through the scleral tissue as fully intact nanoparticles. In certain embodiments, nanoparticles specifically refer to agent-loaded nanoparticles having a volume average diameter from 50 nm to 2000 nm, or from 50 nm to 500 nm, or from 50 nm to 200 nm, or from 50 nm to 150 nm. As used herein, the nanoparticle encompasses nanospheres, nanocapsules, nanoparticles, and nanorods, unless specified otherwise. A nanoparticle may be of composite construction and is not necessarily a pure substance; it may be spherical or any other shape.

“Ocular region” or “ocular site” means any area of the eye, including the anterior and posterior segment of the eye, and which generally includes, but is not limited to, any functional (e.g., for vision) or structural tissues found in the eyeball, or tissues or cellular layers that partly or completely line the interior or exterior of the eyeball. Ocular regions include the anterior chamber, the posterior chamber, the vitreous cavity, the choroid, the suprachoroidal space, the subretinal space, the conjunctiva, the subconjunctival space, the episcleral space, the intracorneal space, the epicorneal space, the sclera, the pars plana, surgically-induced avascular regions, the macula, and the retina.

“Ocular condition” means a disease, ailment or condition which affects or involves the eye or one of the parts or regions of the eye. Broadly speaking the eye includes the eyeball and the tissues and fluids which constitute the eyeball, the periocular muscles (such as the oblique and rectus muscles) and the portion of the optic nerve which is within or adjacent to the eyeball.

A “therapeutically effective amount” refers to a quantity of a specified agent sufficient to achieve a desired effect in a subject being treated with that agent. Ideally, a therapeutically effective amount of an agent is an amount sufficient to inhibit or treat the disease or condition without causing a substantial cytotoxic effect in the subject. The therapeutically effective amount of an agent will be dependent on the subject being treated, the severity of the affliction, and the manner of administration of the therapeutic composition. For example, a “therapeutically effective amount” may be a level or amount of agent needed to treat an ocular condition, or reduce or prevent ocular injury or damage without causing significant negative or adverse side effects to the eye or a region of the eye

“Treatment” refers to a therapeutic intervention that ameliorates a sign or symptom of a disease or pathological condition after it has begun to develop, or administering a compound or composition to a subject who does not exhibit signs of a disease or exhibits only early signs for the purpose of decreasing the risk of developing a pathology or condition, or diminishing the severity of a pathology or condition. As used herein, the term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. The phrase “treating a disease” refers to inhibiting the full development of a disease, for example, in a subject who is at risk for a disease such as glaucoma or age-related macular degeneration. “Preventing” a disease or condition refers to prophylactic administering a composition to a subject who does not exhibit signs of a disease or exhibits only early signs for the purpose of decreasing the risk of developing a pathology or condition, or diminishing the severity of a pathology or condition. In certain embodiments, “treating” means reduction or resolution or prevention of an ocular condition, ocular injury or damage, or to promote healing of injured or damaged ocular tissue

“Pharmaceutical compositions” are compositions that include an amount (for example, a unit dosage) of one or more of the disclosed compounds together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, PA (19th Edition).

### Overview

Disclosed herein is a topical drug delivery platform incorporating a thermoresponsive hydrogel and porous biocompatible metal-organic frameworks (MOFs). The MOF-based topical drug delivery platform shows detectable levels of drug in the vitreous humor and retina to provide an alternative to intravitreal injections.

The composition comprises a thermoresponsive hydrogel and an agent-loaded metal-organic framework (MOF). MOFs are biocompatible crystalline structures that are synthesized by the coordination of metal ions (Fe, Zr, Zn, etc.) to organic linkers. In one aspect, the composition includes agent-loaded metal-organic framework nanoparticles dispersed within a thermoresponsive hydrogel.

Disclosed herein are nanoparticle/hydrogel ocular delivery systems. The delivery systems disclosed herein are noninvasive since a nanoparticle/hydrogel suspension can be self-administered to the lower fornix and removed by the subject (e.g., with tweezers or a saline solution). Current applications for nanoparticles or hydrogels for ocular conditions require injection to the anterior chamber or vitreous by a clinician. In addition, the current clinical standard for glaucoma is topical eye drop medication that lasts a few hours, while for retinal diseases topical eye drop medications do not exist. In contrast, the presently disclosed systems could provide sustained delivery for at least one month.

In some aspects, the composition is applied to the lower fornix (beneath the lower eyelid) as a “daily eye drop” that will comfortably conform to the conjunctival sac and release the medication in a controlled manner. At the end of the dosing period, this transitioned gel/MOF suspension can be removed before administering the next dose as it is non-degradable. This system can be adapted to treat other ocular pathologies. For acute treatment, the gel can be engineered to degrade along with MOFs to eliminate the need for removal.

The composition is intended to increase the likelihood of patient compliance and patient outcome improvements as an alternative to the invasive bimonthly injections. Aspects of the disclosure facilitate use of biocompatible metal-organic frameworks (MOFs) with large surface areas for high loading of proteins, drugs, peptides, or molecules. The presently disclosed composition affords an alternative administration method for treatment of retinal diseases that does not involve frequent invasive administration and can be applied in the convenience of a patients' own home or in a primary care/optometrists' office. The controlled release characteristic of this system over 14 days provides sufficient bioavailability of medication on the ocular surface to penetrate through the thick collagenous tissue of the sclera into the sub-retinal/retinal/vitreous space.

Also disclosed herein is an ocular vaccine delivery system. The eye mucosa has emerged as a promising alternative vaccination route, with the conjunctiva sharing key immunological features with other mucosal tissues. Eye drop vaccination to the conjunctiva could induce both systemic and mucosal immune responses, similar to oral and intranasal vaccines. Furthermore, eye drop vaccines avoid the risk of central nervous system infiltration associated with intranasal routes. However, a major limitation of eyedrop route is that more than 90% of the vaccines are lost during administration due to the rapid washout effect caused by tear drainage, excessive tearing, and improper administration techniques.

To overcome the limitations of current mucosal vaccine delivery systems and leverage the advantages of ocular vaccination, the ocular vaccine delivery system disclosed herein is designed to enhance antigen retention, stability, and controlled release, ultimately improving vaccine efficacy.

### Agent-Loaded MOF Nanoparticles

The MOF is a porous framework that can be loaded with at least one agent.

Illustrative metal ions for MOFs include iron ion, zirconium ion, zinc ion, chromium ion, cadmium ion, copper ion, nickel ion, aluminum ion, cobalt ion, or a combination thereof.

Illustrative organic linkers for MOFs include 2-methylimidazole, 1,4-benzenedicarboxylic acid, 4,4-bipyridine, 1,2-benzenedicarboxylic acid, 1,3-benzenedicarboxylic acid, biphenyl-4-carboxylic acid, or a combination thereof.

In certain embodiments, the agent-loaded metal-organic framework nanoparticles have a volume average diameter that is sufficiently small for diffusing through scleral tissue and degrading in the vitreous.

The agent for inclusion in the delivery systems disclosed may be a therapeutic agent, a diagnostic agent, an imaging agent, a cosmetic agent, or other agents. In one embodiment, the one or more therapeutic agents are useful for treating ocular conditions. Suitable classes of therapeutic agents include, but are not limited to, active agents that lower intraocular pressure, antibiotics (including antibacterials and antifungals), anti-inflammatory agents, chemotherapeutic agents, anti-angiogenic agents, agents that promote nerve regeneration, steroids, immunosuppressants, neuroprotectants, dry eye syndrome treatment agents (e.g., immunosuppressants, anti-inflammatory agents, steroids, comfort agent such as carboxymethyl cellulose), and combinations thereof. In another aspect, the agent is an immunotherapeutic agent. The therapeutic agents described above can be administered alone or in combination to treat ocular conditions.

In one embodiment, the nanoparticles contain one or more active agents that manage (e.g., reduce) elevated IOP or angiogenesis in the eye. Suitable active agents include, but are not limited to, prostaglandins analogs, such as travoprost, bimatoprost, latanoprost, unoprostine, and combinations thereof; and carbonic anhydrase inhibitors (CAL), such as methazolamide, and 5-acylimino- and related imino-substituted analogs of methazolamide; and combinations thereof.

In certain embodiments, the agent may be a chemotherapeutic agent and/or a steroid. In one embodiment, the chemotherapeutic agent is methotrexate. In another embodiment, the steroid is prednisolone acetate, triamcinolone, prednisolone, hydrocortisone, hydrocortisone acetate, hydrocortisone valerate, vidarabine, fluorometholone, fluocinolone acetonide, triamcinolone acetonide, dexamethasone, dexamethasone acetate, loteprednol etabonate, prednisone, methylprednisone, betamethasone, beclometasone, fludrocortisone, deoxycorticosterone, aldosterone, and combinations thereof.

In certain embodiments, the agent may be an immunosuppressant. Illustrative immunosuppressants include pimecrolimus, tacrolimus, sirolimus, cyclosporine, and combinations thereof.

In a further embodiment, the agent may be a beta adrenergic receptor antagonist or an alpha adrenergic receptor agonist.

Illustrative beta adrenergic receptor antagonists include timolol, levobunalol, carteolol, metipranolol, betaxolol, or a pharmaceutically acceptable salt thereof, or combinations thereof. Illustrative alpha adrenergic receptor agonists include brimonidine, apraclonidine, or a pharmaceutically acceptable salt thereof, or combinations thereof. Additional examples of anti-glaucoma agents include pilocarpine, epinephrine, dipivefrin, carbachol, acetazolamide, dorzolamide, brinzolamide, latanoprost, and bimatoprost.

The agent may be an antibiotic. Illustrative antibiotics include, but are not limited to, cephaloridine, cefamandole, cefamandole nafate, cefazolin, cefoxitin, cephacetrile sodium, cephalexin, cephaloglycin, cephalosporin C, cephalothin, cafcillin, cephamycins, cephapirin sodium, cephradine, penicillin BT, penicillin N, penicillin O, phenethicillin potassium, pivampic ulin, amoxicillin, ampicillin, cefatoxin, cefotaxime, moxalactam, cefoperazone, cefsulodin, ceflizoxime, ceforanide, cefiaxone, ceftazidime, thienamycin, N-formimidoyl thienamycin, clavulanic acid, penemcarboxylic acid, piperacillin, sulbactam, cyclosporins, moxifloxacin, vancomycin, and combinations thereof.

The agent may be an inhibitor of a growth factor receptor. Suitable inhibitors include, but are not limited to, inhibitors of Epidermal Growth Factor Receptor (EGFR), such as AG1478, and EGFR kinase inhibitors, such as BIBW 2992, erlotinib, gefitinib, lapatinib, and vandetanib, and inhibitors of vascular endothelial growth factor (VEGF) such as ranibizumab, bevacizumab, aflibercept, brolucizumab, and faricimab.

Also disclosed herein is an ocular immunotherapeutic agent delivery system that includes immunotherapeutic agent loaded metal-organic framework nanoparticles dispersed within a thermoresponsive hydrogel.

Illustrative immunotherapeutic agents include a vaccine antigen. Illustrative vaccine antigens include ovalbumin. In certain embodiments, a vaccine antigen may be incorporated into metal-organic framework nanoparticle and a vaccine adjuvant may be incorporated independently into metal-organic framework nanoparticle. Illustrative vaccine adjuvants include cholera toxin B subunit.

In certain embodiments, the amount of agent loaded into the nanoparticles may range from 1 μg to 1000 μg, and most particularly, 400 μg to 600 μg agent per mg of nanoparticles.

The nanoparticles can be administered alone or in combination with microparticles containing a second drug that lowers IOP, reduces inflammation, or is neuroprotective.

Aspects of the disclosure concern the use of MOFs loaded with a therapeutic agent, such as a clinically approved neovascular age-related macular degeneration (nvAMD) medication. In particular aspects, the therapeutic agent is ranibizumab (Lucentis C); however, other drugs, proteins, peptides, or molecules of interest can be encapsulated into MOFs. In some aspects, the MOFs provide at least 14 days of protein release, but modifications can be made to tune the release profile to better fit the desired profile for the molecule being loaded.

### Gels

In some aspects, the MOFs are suspended in a poly-N-isopropylacrylamide (pNIPAAm) thermoresponsive hydrogel that has been engineered to have a sol-gel transition at temperatures above 32° C. In some aspects, the thermoresponsive gel is a gel as described in U.S. Publication No. 20220211632, the relevant portion of which is incorporated herein by reference. The application of this gel/MOF suspension provides long-term delivery of nvAMD medication as a replacement for the current clinical standard of treatment of intravitreal injections bimonthly.

In certain embodiments, the hydrogel may respond to external stimulus (e.g., physiological conditions) such as changes in ion concentration, pH, temperature, glucose, shear stress, or a combination thereof. Illustrative hydrogels include polyacrylamide (e.g., poly-N-isopropylacrylamide), silicon hydrogels like those used in contact lenses, polyethylene oxide/polypropylene oxide or combinations of the two (e.g., Pluronics hydrogel or Tectronics hydrogel), butyl methacrylate, polyethylene glycol diacrylate, polyethylene glycol of varying molecular weights, polyacrylic acid, poly methacrylic acid, poly lactic acid, poly(tetramethyleneether glycol), poly(N,N′-diethylaminoethyl methacrylate), methyl methacrylate, and N,N′-dimethylaminoethylmethacrylate. In certain embodiments, the hydrogel is a thermoresponsive hydrogel.

In certain embodiments, the thermoresponsive hydrogel has a lower critical solution temperature (LCST) below body temperature. The thermoresponsive hydrogel remains fluid below physiological temperature (e.g., 37° C. for humans) or at or below room temperature (e.g., 25° C.), solidify (into a hydrogel) at physiological temperature, and are biocompatible. For example, the thermoresponsive hydrogel may be a clear liquid at a temperature below 34° C. which reversibly solidifies into a gelled composition at a temperature above 34° C. Generally, the LCST-based phase transition occurs upon warming in situ as a result of entropically-driven dehydration of polymer components, leading to polymer collapse. Various naturally derived and synthetic polymers exhibiting this behavior may be utilized. Natural polymers include elastin-like peptides and polysaccharides derivatives, while notable synthetic polymers include those based on poly(n-isopropyl acrylamide) (PNIPAAm), poly(N,N-dimethylacrylamide-co-N-phenylacrylamide), poly(glycidyl methacrylate-co-N-isopropylacrylamide), poly(ethylene oxide)-b-poly(propylene oxide)-b-poly(ethylene oxide), poly(ethylene glycol)-polyester copolymer, and amphiphilic block copolymers. The structure of PNIPAAm, containing both hydrophilic amide bonds and hydrophobic isopropyl groups, leads to a sharp phase transition at the LCST. Studies suggest that the average number of hydrating water molecules per NIPAAm group falls from 11 to about 2 upon the hydrophobic collapse above the LCST (32-34° C.). In certain embodiments, the amphiphilic block copolymer comprises a hydrophilic component selected from poly ethylene oxide (PEO), poly vinyl alcohol (PVA), poly glycolic acid (PGA), poly(N-isopropylacrylamide), poly(acrylic acid) (PAA), poly vinyl pyrrolidone (PVP) or mixtures thereof, and a hydrophobic component selected from polypropylene oxide (PPO), poly(lactic acid) (PLA), poly(lactic acid co glycolic acid) (PLGA), poly (.beta.-benzoyl L-aspartate) (PBLA), poly (.gamma.-benzyl-L-glutamate) (PBLG), poly (aspartic acid), poly (L-lysine), poly(spermine), poly (caprolactone) or mixtures thereof. Examples of such amphiphilic block copolymers include (PEO) (PPO) (PEO) block copolymers (PEO/PPO), and poly (lactic acid co glycolic acid) block copolymers (PLGA), such as (PEO) (PLGA) (PEO) block copolymers.

In certain embodiments, the hydrogel is non-biodegradable (e.g., PNIPAAm). In other embodiments, the hydrogel is biodegradable. For example, biodegradable NIPAAm-based polymers can be made by conjugating the PNIPAAm with natural biodegradable segments such as MMP-susceptible peptide, gelatin, collagen, hyaluronic acid and dextran. Copolymers formed from NIPAAm and monomers with degradable side chains comprise another category of NIPAAm-based bioabsorbable, thermoresponsive hydrogels. Hydrolytic removal of hydrophobic side chains increases the hydrophilicity of the copolymer, raising the LCST above body temperature and making the polymer backbone soluble. Due to the relative simplicity of the synthetic process, the most investigated biodegradable monomers have been HEMA-based monomers, such as 2-hydroxyethyl methacrylate-polylactide (HEMA-PLA) (Lee, B. H.; et al. Macromol. Biosci. 2005, 5, 629-635; and Guan, J., et al. Biomacromolecules 2008, 9, 1283-92), 2-hydroxyethyl methacrylate-polycaprolactone (HEMA-PCL) (Wang, T., et al. Eur. J. Heart Fail 2009, 11, 14-19 and Wu, D., et al. ACS Appl. Mater. Interf. 2009, 2, 312-327) and 2-hydroxyethyl methacrylate-polytrimethylene carbonate (HEMA-PTMC) (Fujimoto, K. L., et al. Biomaterials 2009, 30, 4357-4368 and Wang, F., et al. Acta Biomater. 2009, 5, 2901). However, the backbone remnant following hydrolysis, HEMA, presents hydroxyethyl side groups (—CH.sub.2CH.sub.2-OH), which have a relatively limited effect on remnant polymer hydrophilicity (Cui, Z., et al. Biomacromolecules 2007, 8, 1280-1286). In previous studies, such hydrogels have been found to be either partially bioabsorbable (Wu, D., et al. ACS Appl. Mater. Interf. 2009, 2, 312-327) or completely bioabsorbable, but have required the inclusion of considerably hydrophilic co-monomers such as acrylic acid (AAc) in the hydrogel synthesis (Fujimoto, K. L.; et al. Biomaterials 2009, 30, 4357-4368; Wang, F., et al. Acta Biomater. 2009, 5, 2901; and Guan, J., et al. Biomacromolecules 2008, 9, 1283-92).

In a further embodiment, the thermoresponsive hydrogel degrades and dissolves at physiological conditions in a time-dependent manner. The copolymer and its degradation products typically are biocompatible. According to one embodiment, the copolymer consists essentially of N-isopropylacrylamide (NIPAAm) residues (a residue is a monomer incorporated into a polymer), hydroxyethyl methacrylate (HEMA) residues and methacrylate-polylactide (MAPLA) macromer residues as disclosed in U.S. Patent Publ. 2012/0156176, which is incorporated herein by reference. Alternately, the copolymer consists essentially of N-isopropylacrylamide residues, acrylic acid (AAc) residues, and hydroxyethyl methacrylate-poly (trimethylene carbonate) (HEMAPTMC) macromer residues as disclosed in U.S. Patent Publ. 2012/0156176, which is incorporated herein by reference.

The base precursor (e.g., a prepolymer, oligomer and/or monomer) for the hydrogel, cross linkers, and initiators are mixed together and allowed to polymerize for a predefined period of time (from 1 h to 24h typically) to form the hydrogel. The hydrogel is then washed to remove any excess initiator or unreacted materials. The hydrogel at this stage is a liquid (e.g., in the form of an aqueous solution) at room temperature until it is ready for use. The microparticles can be added in before, after, or during the polymerization of the hydrogel (adding microparticles in before or during polymerization results in a slighter faster initial drug release rate) to form a suspension of solid microparticles in hydrogel. The amount of microparticles loaded into the hydrogel may vary. For example, there may be up to 10 mg, more particularly 1 to 5 mg microparticles per microliter hydrogel. In certain embodiments, the microparticles are homogeneously dispersed within the hydrogel. Optional components can be added that allow for easier visualization of the hydrogel/microparticle suspension such as sodium fluorescein or other fluorescent molecules such as FITC, rhodamine, or AlexaFluors or dyes such as titanium dioxide. The water content of the swollen hydrogel at room temperature may be 50-80%. The water content of the hydrogel after it gels in situ in the eye may be 1-10%.

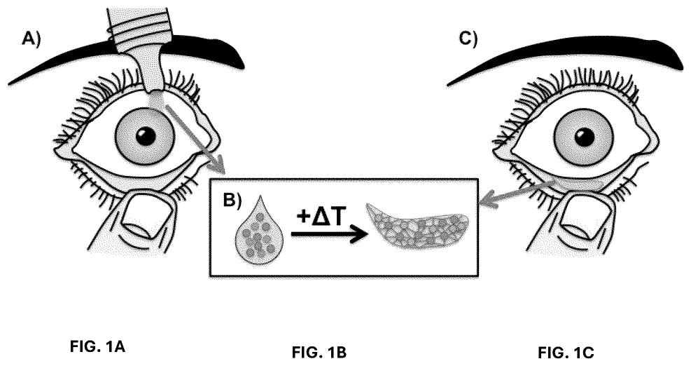

Upon ocular administration of the nanoparticle/hydrogel liquid suspension, the nanoparticle/hydrogel system releases water and can become an opaque solid gel member. The gelled member may be sufficiently firm that it can be manipulated with tweezers. FIG. 1A depicts administration of an eye drop 1 comprising the nanoparticle/hydrogel liquid suspension, gelling of the suspension to form a polymeric crosslinked matrix 2 that encapsulates the agent-loaded nanoparticles (FIG. 1B), and positioning of the resulting gelled member 3 in the lower fornix of the eye (FIG. 1C). In one particular embodiment, a thermoresponsive hydrogel carrier for the agent-loaded nanoparticles has been developed and characterized that will allow patients to apply a liquid suspension (containing the release system) topically to their eye as they would an aqueous eye drop-based medication (FIG. 1A). When the drop collects in the conjunctival cul-de-sac, the liquid warms to body temperature and thermoresponsive hydrogel de-swells, forming a stable, opaque gel (FIG. 1B). The drop also appears to naturally conform to the shape of the inferior fornix during the gelation (FIG. 1C) promoting retention of the system and continuous delivery of agent to the eye via the embedded, sustained agent nanoparticle formulation. Furthermore, removal of the gelled drop would be as simple as flushing the eye with cold saline, unlike intravitreal or subconjunctival implants that require removal by a clinician. This formulation should lower IOP and increase bioavailability compared to topical eye drops. This new delivery formulation could also serve as a modular platform for local administration of not only a variety of glaucoma medications (including BT), but a whole host of other ocular therapeutics as well.

The shape of the gelled member 3 may vary and is dependent on the anatomy of the ocular structure. Typically, the gelled member 3 spreads out into an elongate, thin film of gel, but it may assume a more cylindrical shape. In certain embodiments, the gelled film may have a thickness of 10 to 1000, more particularly 100 to 300 μm. The gel can be manipulated as it undergoes phase transitioning into a desired shape. In certain embodiments, the gelled member may retain pliability to a certain extent. In certain embodiments, the gelled member 3 may have a residence time in the lower fornix of at least five days, more particularly at least 10 days, and most particularly at least 30 days.

The nanoparticle/hydrogel system disclosed herein may provide for sustained release of an agent. The agent release can be linear or non-linear (single or multiple burst release). In certain embodiments, the agent may be released without a burst effect. For example, the sustained release may exhibit a substantially linear rate of release of the therapeutic agent in vivo over a period of at least 5 days, more particularly at least 10 days, and most particularly at least 30 days. By substantially linear rate of release it is meant that the therapeutic agent is released at a rate that does not vary by more than about 20% over the desired period of time, more usually by not more than about 10%. It may be desirable to provide a relatively constant rate of release of the agent from the delivery system over the life of the system. For example, it may be desirable for the agent to be released in amounts from 0.1 to 100 μg per day, more particularly 1 to 10 μg per day, for the life of the system. However, the release rate may change to either increase or decrease depending on the formulation of the polymer nanoparticle and/or hydrogel. In certain embodiments, the delivery system may release an amount of the therapeutic agent that is effective in providing a concentration of the therapeutic agent in the eye in a range from 1 ng/ml to 200 μg/ml, more particularly 1 to 5 μg/ml. The desired release rate and target drug concentration can vary depending on the particular therapeutic agent chosen for the drug delivery system, the ocular condition being treated, and the subject's health.

In certain embodiments, the agent release is dependent on degradation of the polymer nanoparticles. As the crystalline chains break up, the agent can diffuse out of the initial crystalline nanoparticle matrix where it will eventually reach the hydrogel matrix. At that point, the hydrogel may partially slow down release of the agent but diffusion through the hydrogel is significantly faster than degradation of the crystalline matrix. Thus the limiting factor in agent release is degradation of the crystalline matrix.

The nanoparticle/hydrogel composition may be administered in the form of a liquid eye drop. The eye drop(s) may be administered to any ocular structure, but is preferably administered to the lower fornix. The eye drops may be self-administered by the subject. The eye drop will conform comfortably to the conjunctival sac and release the loaded agent. The eye drop may be administered on a regimen wherein the interval between successive eye drops is greater than at least one day (although in certain embodiments the eye drop may be administered once daily or more than once daily). For example, there may be an interval of at least 5 days, at least one week, or at least one month between administrations of an eye drop(s). In preferred embodiments, the disclosed eye drops may be used for sustained monthly delivery of medication as a replacement for the current clinical standard of monthly intravitreal injection administration. At the end of the desired administration period, the gelled member can be removed from the eye (for example, via a tweezer or flushing out). In certain embodiments, the hydrogel may be biodegradable so that there is no need to remove the gelled member (this embodiment may be most useful for treating an acute condition). This system disclosed herein not only provides a non-invasive administration method (thereby increasing the likelihood of patient compliance and recovery/prevention of worsening symptoms), it does so while avoiding clinician involvement for administration by being completely noninvasive.

The nanoparticle/hydrogel disclosed herein may include an excipient component, such as effective amounts of buffering agents, and antioxidants to protect a drug (the therapeutic agent) from the effects of ionizing radiation during sterilization. Suitable water soluble buffering agents include, without limitation, alkali and alkaline earth carbonates, phosphates, bicarbonates, citrates, borates, acetates, succinates and the like, such as sodium phosphate, citrate, borate, acetate, bicarbonate, carbonate and the like. These agents are advantageously present in amounts sufficient to maintain a pH of the system of between about 2 to about 9 and more preferably about 4 to about 8. As such the buffering agent may be as much as about 5% by weight of the total system. Suitable water soluble preservatives include sodium bisulfite, sodium bisulfate, sodium thiosulfate, ascorbate, benzalkonium chloride, chlorobutanol, thimerosal, phenylmercuric acetate, phenylmercuric borate, phenylmercuric nitrate, parabens, methylparaben, polyvinyl alcohol, benzyl alcohol, phenylethanol and the like and mixtures thereof. These agents may be present in amounts of from 0.001 to about 5% by weight and preferably 0.01 to about 2% by weight.

The nanoparticle/hydrogel system disclosed herein may be useful to treat a variety of ocular conditions, both chronic and acute. Illustrative ocular conditions include: maculopathies/retinal degeneration: macular degeneration, including age related macular degeneration (AMD), such as non-exudative age related macular degeneration and exudative age related macular degeneration, choroidal neovascularization, retinopathy, including diabetic retinopathy, acute and chronic macular neuroretinopathy, central serous chorioretinopathy, and macular edema, including cystoid macular edema, and diabetic macular edema. Uveitis/retinitis/choroiditis: acute multifocal placoid pigment epitheliopathy, Behcet's disease, birdshot retinochoroidopathy, infectious (syphilis, lyme, tuberculosis, toxoplasmosis), uveitis, including intermediate uveitis (pars planitis) and anterior uveitis, multifocal choroiditis, multiple evanescent white dot syndrome (MEWDS), ocular sarcoidosis, posterior scleritis, serpignous choroiditis, subretinal fibrosis, uveitis syndrome, and Vogt-Koyanagi-Harada syndrome. Vascular diseases/exudative diseases: retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angitis, sickle cell retinopathy and other hemoglobinopathies, angioid streaks, familial exudative vitreoretinopathy, Eales disease. Traumatic/surgical: sympathetic ophthalmia, uveitic retinal disease, retinal detachment, trauma, laser, PDT, photocoagulation, hypoperfusion during surgery, radiation retinopathy, bone marrow transplant retinopathy. Proliferative disorders: proliferative vitreal retinopathy and epiretinal membranes, proliferative diabetic retinopathy. Infectious disorders: ocular histoplasmosis, ocular toxocariasis, presumed ocular histoplasmosis syndrome (PONS), endophthalmitis, toxoplasmosis, retinal diseases associated with HIV infection, choroidal disease associated with HIV infection, uveitic disease associated with HIV Infection, viral retinitis, acute retinal necrosis, progressive outer retinal necrosis, fungal retinal diseases, ocular syphilis, ocular tuberculosis, diffuse unilateral subacute neuroretinitis, and myiasis. Genetic disorders: retinitis pigmentosa, systemic disorders with associated retinal dystrophies, congenital stationary night blindness, cone dystrophies, Stargardt's disease and fundus flavimaculatus, Bests disease, pattern dystrophy of the retinal pigmented epithelium, X-linked retinoschisis, Sorsby's fundus dystrophy, benign concentric maculopathy, Bietti's crystalline dystrophy, pseudoxanthoma elasticum. Retinal tears/holes: retinal detachment, macular hole, giant retinal tear. Tumors: retinal disease associated with tumors, congenital hypertrophy of the RPE, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, intraocular lymphoid tumors. Miscellaneous: punctate inner choroidopathy, acute posterior multifocal placoid pigment epitheliopathy, myopic retinal degeneration, acute retinal pigment epithelitis and the like.

In certain embodiments, the ocular conditions include glaucoma, chronic dry eye, keratitis, post-operative inflammation, conjunctivitis, and bacterial or fungal infections.

Also disclosed herein are methods of controlling IOP in a subject using the above-described drug delivery systems. In various embodiments, IOP is maintained at or below about 22 mmHg. The drug may be released such that the concentration of the drug is approximately constant over a period of at least one day. In other embodiments, the above methods control the IOP for a period of at least 1 day, 2 days, 3 days, or 1 week.

### Overview of Several Embodiments

Disclosed herein is a composition, comprising: a thermoresponsive gel; and a metal-organic framework material (MOF).

In any or all aspects, the thermoresponsive gel is a poly-N-isopropylacrylamide (pNIPAAm) gel.

In any or all aspects, the MOF comprises a metal ion and an organic linker.

In any or all aspects, the metal ion is selected from an iron ion, zirconium ion, zinc ion, chromium ion, cadmium ion, copper ion, nickel ion, or a combination thereof.

In any or all aspects, the MOF is a zeolitic imidazolate.

In any or all aspects, the MOF comprises zinc and 2-methylimidazole.

In any or all aspects, the MOF is ZIF-8.

In any or all aspects, the composition further comprises a therapeutic agent.

In any or all aspects, the therapeutic agent is a therapeutic agent that treats an eye disease or an eye disorder.

In any or all aspects, the therapeutic agent is a neovascular age-related macular degeneration medication.

Also disclosed is a method, comprising administering a composition according to any or all of the above aspects into the eye of a subject.

In any or all aspects, the composition is formulated as a topical eye drop.

In any or all aspects, the composition is administered to the lower fornix of the eye of the subject.

Also disclosed is a method of making a composition for administration to an eye of a subject, the method comprising combining metal-organic framework (MOF) particles with a thermoresponsive gel to form the composition.

In any or all aspects, the method further comprises combining a therapeutic agent with the MOF particles.

### EXAMPLES

### Example 1

Zeolitic imidazolate framework (ZIF-8) was synthesized in a purely aqueous solution of Zn²⁺ and 2-methylimidazole at a molar ratio of 1:16 poly (lactic-co-glycolic acid). PLGA microparticles (MP) were synthesized, for comparative purposes, using a double emulsion solvent-evaporation protocol. Ovalbumin was loaded into each type of particle (MOF particles and MP particles, separately) at a mass of 10-40 mg and then the particles were incorporated into a thermoresponsive gel. The thermoresponsive gel was made according to the following protocol: Make 100 mg/mL APS in milliQ water; weigh 500 mg NIPAAm and add to 15 mL Falcon tube; add 9.44 mL milliQ water-vortex; add 50 μL 100 mg/mL APS-vortex; add 20 UL TEMED-vortex; store at 2-5° C. fridge for 24 hours; wash 2× to remove unreacted chemicals; and rehydrate using PEG 200 to desired % w/v.

Biodistribution studies involved healthy New Zealand white rabbits (n=5). The right eye of the rabbits was given a 100 μL of gel with the MOF and PLGA MP particles, independently, and a 50 μL injection of 10 mg/mL ovalbumin solution, for comparative purposes. The left eye was the experimental control. At 1 and 3 days, rabbits were sacrificed, and both eyes were removed. Aqueous humor, vitreous humor, retina-choroid and plasma were collected, and time-dependent concentrations were quantified with ELISA.

After one administration of the topical delivery system, ovalbumin is detectable in the aqueous humor, vitreous humor, retina-choroid, and plasma. Interestingly, ovalbumin concentrations in the retina-choroid are 9× the concentration in the vitreous humor (˜1 ng/ml). However, these concentrations are 10⁵ lower than what is seen with intravitreal injections after 3 days. The results suggest that the topical delivery platform can increase the ocular surface residence time and control the release of ovalbumin.

### Example 2

ZIF-8 was prepared using a modified protocol (Pan 2011) using an aqueous reaction of metal nodes and organic linkers. Ovalbumin (test agent, acting as a surrogate agent for other anti-VEGF proteins) was loaded (1 gram) into the organic linker aqueous solution, and the metal node and organic linker+ovalbumin solution were mixed and allowed to undergo reaction to form a cloudy solution. This type of loading by adding agent in with reactants is called “one-pot” synthesis. After 5 minutes, the reaction solution was centrifuged, washed with water, then lyophilized for 48 hours.

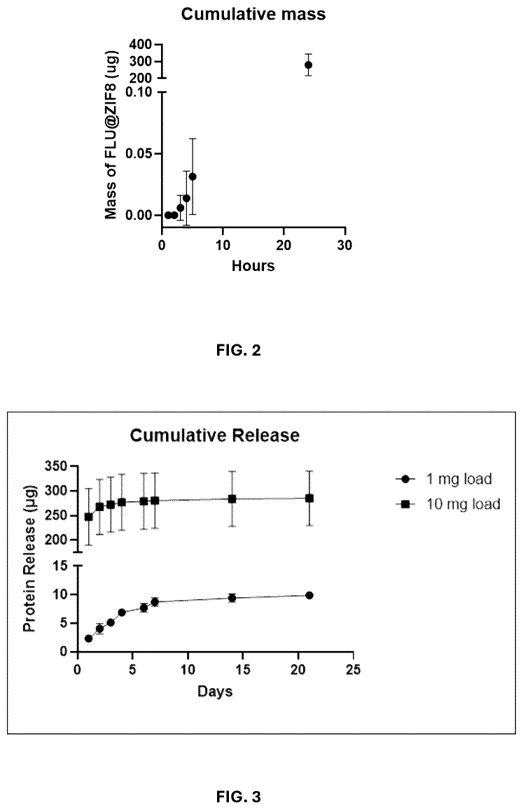

For the permeability study, porcine eyes were acquired from a local butcher shop (Thoma Meat Market, Saxonburg, PA) and a Franz Diffusion cell apparatus was used to assess scleral permeability. Porcine scleral was removed and placed to cover the diffusion cell entirely. Two groups were assessed: one control group (1 mL of water, n=3) and the ZIF-8 group (20 mg/mL ZIF-8, n=3). ZIF-8 was modified to encapsulate fluorescein and the physical color of the material changed from white to red (indicating encapsulation of fluorescein). The two experimental groups were applied to the scleral tissue and allowed to permeate through the tissue unperturbed. Sample points were taken at every hour for the first 5 hours, then another sample was taken at 24 hours. The results were measured using a microplate spectrometer and measured for fluorescence at an excitation wavelength of 490 nm and an emission wavelength of 519 nm and compared to a standard curve previous created. Due to the stability of ZIF-8 in water, the fluorescein detected in the sample media is expected to be stable, fluorescein-loaded ZIF-8 and a cumulative mass over the 24-hour experiment of 279.5 μg was quantified (FIG. 2). In addition, although no samples were taken past 24 hours, we did allow for longer exposure of ZIF-8 with the scleral surface and witnessed continued permeability at 72 hours post-administration.

Due to the relative size of the ZIF8 MOFs (˜100 nm), they are fully intact once they reach the posterior chamber and change the mechanism of release compared to the mechanism of the U.S. Publication No. 20220211632 PLGA microparticle+gel system. Instead of degrading at the ocular surface and the protein diffusing through the scleral tissue, the particle itself is diffusing through the scleral tissue and degrading in the vitreous where the protein can easily diffuse to the retina and other posterior tissue. The U.S. Publication No. 20220211632 PLGA+gel system microparticles are 104 times larger (˜10 μm) and cannot pass through the sclera at that size.

Additional differences between the ZIF-8 MOF and U.S. Publication No. 20220211632 PLGA microparticle+gel system involve their loading and release.

For the U.S. Publication No. 20220211632 PLGA microparticle+gel system, 10 milligrams of ovalbumin is loaded into the PLGA microparticles with a loading capacity of 47.13 μg/mg. The synthesis protocol uses a double emulsion solvent evaporation which is a water and oil system to create polymeric particles. This process is limited by the amount of drug that can be loaded into the first water phase due to the solubility of the drug or protein, and the relative instability of the oil and water phases. The release profile of these particles has a relatively high burst release in the first day and levels off over 1 week.

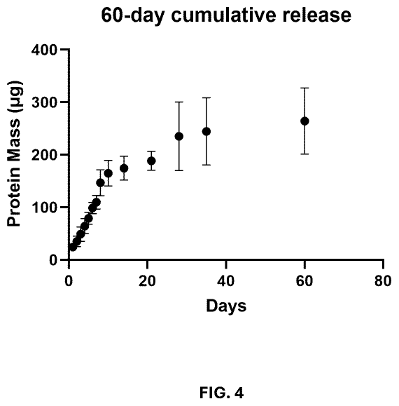

Conversely, ZIF-8 MOFs are loaded with 1 gram of ovalbumin and have a loading capacity of 463.4 μg/mg—this is mainly due to the formulation procedure that allows for direct incorporation of the drug or agent into the particle and solubility is not an issue. The release profile is more linear during a 1-week timeframe. Cumulatively, both particles release 250-300 μg in the first week and can continue releasing over several weeks. While PLGA typically degrades within 30 days (FIG. 3), we have continued release of ovalbumin from ZIF-8 MOFs at +90 days releasing a total of ˜400 μg (FIG. 4).

### Example 3

Ovalbumin (OVA) and cholera toxin B subunit (CTB) will be encapsulated within ZIFs and incorporated into a poly(N-isopropylacrylamide) (PNIPAAm)-based thermoresponsive hydrogel. The system will be characterized for antigen encapsulation efficiency, stability, and controlled release. In vitro biocompatibility will be assessed using human corneal and conjunctival epithelial cells. In vivo studies in healthy rabbits will evaluate immune responses via secretory IgA (sIgA) in tears and OVA-specific IgG/IgA in serum. Additionally, an allergic airway rabbit model will be utilized to assess potential inflammatory effects. The ZIF-hydrogel is expected to enhance antigen retention, promote sustained immune activation, and reduce the need for multiple doses, while ensuring ocular biocompatibility. Minimal inflammatory responses in allergic rabbits will confirm its safety. This innovative vaccine platform has the potential to transform mucosal immunization by offering a non-invasive, stable, and effective alternative to existing methods, with significant implications for global vaccine accessibility and distribution.

ZIF particles will be synthesized using a one-pot method. Briefly, a zinc salt (e.g., zinc nitrate) will be mixed with an excess of 2-methylimidazole, which is the organic ligand. OVA-encapsulated ZIF and CTB-encapsulated ZIF will be synthesized separately by adding OVA or CTB to the 2-methylimidazole solution during synthesis. Encapsulation efficiency and structural integrity will be analyzed using dynamic light scattering (DLS), Fourier-transform infrared spectroscopy (FTIR), X-ray diffraction (XRD), and protein quantification assays (BCA, ELISA).

The thermoresponsive hydrogel will be prepared by rehydrating poly(N-isopropylacrylamide) (PNIPAAm) in water with polyethylene glycol (PEG). The ratio of PNIPAAm to PEG will be varied, and the concentration of PNIPAAm will be adjusted to 5%, 10%, and 15% to refine the phase transition properties and viscosity. OVA- and CTB-encapsulated ZIF particles will be incorporated into the hydrogel at varying concentrations (2%, 5%, and 10%) for further characterization.

The sustained release profiles of OVA and CTB from ZIFs and ZIF within the hydrogel will be assessed using ELISA and HPLC over time in simulated tear fluid.

To assess thermostability, OVA-ZIF particles will be stored at 60° C., 25° C., and 4° C. for one month. OVA stability will be evaluated by comparing the in vitro release profiles under different storage conditions. A non-encapsulated OVA solution will serve as a control.

The cytotoxicity of the ZIF-hydrogel formulation will be evaluated using human corneal and conjunctival epithelial cell lines via an MTT assay.

In view of the many possible embodiments to which the principles of the disclosed compositions and methods may be applied, it should be recognized that the illustrated embodiments are only examples of the compositions and methods.

What is claimed is:

1. A composition comprising agent-loaded metal-organic framework nanoparticles dispersed within a thermoresponsive hydrogel, wherein the agent is an agent for treating an ocular condition and the composition is configured for sustained topical ocular release.

2. The composition of claim 1, wherein the composition is a liquid that can be administered to a subject in the form of an eye drop.

3. The composition of claim 1, wherein the agent is an agent that inhibits angiogenesis, the nanoparticles are biodegradable, and the hydrogel is not biodegradable.

4. The composition of claim 1, wherein the agent-loaded metal-organic framework nanoparticles have a volume average diameter from 50 nm to 2000 nm.

5. The composition of claim 1, wherein the agent-loaded metal-organic framework nanoparticles have a volume average diameter from 50 nm to 500 nm.

6. The composition of claim 1, wherein the agent-loaded metal-organic framework nanoparticles have a volume average diameter from 50 nm to 150 nm.

7. The composition of claim 1, wherein the agent-loaded metal-organic framework nanoparticles have a volume average diameter that is sufficiently small for diffusing through scleral tissue and degrading in the vitreous.

8. The composition of claim 1, wherein the agent-loaded metal-organic framework nanoparticles have an agent loading of 1 μg to 1000 μg agent, per mg of nanoparticles.

9. The composition of claim 1, wherein the agent-loaded metal-organic framework nanoparticles have an agent loading of 1 μg to 600 μg agent, per mg of nanoparticles.

10. The composition of claim 1, wherein the agent-loaded metal-organic framework nanoparticles have an agent loading of 400 μg to 600 μg agent, per mg of nanoparticles.

11. The composition of claim 1, wherein the thermoresponsive gel comprises poly-N-isopropylacrylamide.

12. The composition of claim 1, wherein the metal-organic framework comprises a metal ion and an organic linker, and the metal ion is selected from an iron ion, zirconium ion, zinc ion, chromium ion, cadmium ion, copper ion, nickel ion, or a combination thereof.

13. The composition of claim 1, wherein the metal-organic framework is a zeolitic imidazolate.

14. The composition of claim 1, wherein the metal-organic framework comprises zinc and 2-methylimidazole.

15. The composition of claim 1, wherein the metal-organic framework is ZIF-8.

16. The composition of claim 1, wherein the agent is a therapeutic agent that treats an eye disease or an eye disorder.

17. The composition of any one of claim 1, wherein the agent is a neovascular age-related macular degeneration medication.

18. A method comprising:
combining an agent with metal-organic framework nanoparticles; and
combining the resulting agent-loaded metal-organic framework nanoparticles with a thermoresponsive hydrogel to form a composition.

19. The method of claim 18, wherein the composition is a liquid that can be administered to a subject in the form of an eye drop.

20. A method for ocular delivery of an agent comprising administering the agent at the lower fornix of an eye in a subject, wherein the method comprises topically delivering to an eye a liquid hydrogel comprising agent-loaded metal-organic framework nanoparticles, and permitting the liquid hydrogel to form in situ a gelled, sustained release structure residing in the lower fornix of the eye.

21. The method of claim 20, wherein the agent-loaded metal-organic framework nanoparticles diffuse through scleral tissue and degrade in the vitreous.

22. A composition comprising immunotherapeutic agent-loaded metal-organic framework nanoparticles dispersed within a thermoresponsive hydrogel, wherein the composition is configured for delivering the immunotherapeutic agent.

23. The composition of claim 22, wherein the immunotherapeutic agent is a vaccine antigen.

24. The composition of claim 23, wherein the composition further comprises vaccine adjuvant-loaded metal-organic framework nanoparticles dispersed within the thermoresponsive hydrogel.
